(19) United States
(12) Patent Application Publication
(10) Pub. No.: US 2020/0027568 A1
(43) Pub. Date: Jan. 23, 2020

(54) Physician House Call Portal

(71) Applicant: West Affum Holdings Corp., Grand Cayman (KY)

(72) Inventors: Phillip D. Foshee, Jr., Woodinville, WA (US); Zoie R. Engman, Kirkland, WA (US); Daniel J. Finney, Woodinville, WA (US)

(21) Appl. No.: 16/514,907

(22) Filed: Jul. 17, 2019

Related U.S. Application Data
(60) Provisional application No. 62/699,167, filed on Jul. 17, 2018.

(51) Int. Cl. G16H 80/00; G06F 13/20; H04L 29/08; A61B 5/00; A61B 5/021; A61B 7/02; A61B 1/227; A61B 5/0428

(52) U.S. Cl. CPC G16H 80/00; G06F 13/20; H04L 67/12; H04L 67/141; A61B 5/7465; G06F 2213/40; A61B 5/0022; A61B 5/021; A61B 7/02; A61B 1/227; A61B 5/0428; A61B 5/0006

(57) ABSTRACT

A physician house call portal comprises a processor and a memory coupled to the processor to store a patient station application, a communication system to communicate with a physician station via a network, an input/output (I/O) system coupled with an input device and an output device to allow a patient to communicate with a medical practitioner using the physician station, and a medical sensor to obtain a patient physiological parameter using the patient station application. Instructions in the memory configure the processor to allow the medical practitioner to perform an examination of the patient using the medical sensor.

### CROSS-REFERENCE TO RELATED APPLICATIONS

The present application claims the benefit of U.S. Provisional Application No. 62/699,167 (C00003604.USP2) filed Jul. 17, 2018. Said Application No. 62/699,167 is hereby incorporated herein by reference in its entirety.

### BACKGROUND

Physicians have ever greater demands on their time as patient loads increase and the amount of time a physician is able to spend with a patient decreases. In rural communities, there may not be enough physicians in the area to adequately service the needs of the local patients. At one time, physicians performed house calls in which patient-physician meetings were conducted at the patient's home. In recent times, physician house calls have been phased out in order to allow the physician to service more patients per hour at a single location. Now, instead of the physician traveling to the patient's home, rural patients are forced to travel great distances to visit the physician's office even just for routine examinations. Advances in telemedicine allow the patient-physician meeting paradigm to shift back to the house call model to enable physicians to perform medical evaluations of remotely located patients without requiring either the patient or the physician to travel long distances.

### DESCRIPTION OF THE DRAWING FIGURES

Claimed subject matter is particularly pointed out and distinctly claimed in the concluding portion of the specification. However, such subject matter may be understood by reference to the following detailed description when read with the accompanying drawings in which:

FIG. 1 is a diagram of a physician house call system including a patient station and a physician station in accordance with one or more embodiments.

FIG. 2 is a diagram of a physician house call portal including a patient station to obtain one or more patient parameters in accordance with one or more embodiments.

FIG. 3 is a diagram of a physician house call portal with which a patient can view a remotely located medical practitioner in accordance with one or more embodiments.

FIG. 4 is a diagram of a physician house call portal with which a medical practitioner can view a remotely located patient in accordance with one or more embodiments.

FIG. 5 is a diagram of example components of a physician house call portal including electrocardiogram (ECG) sensors in accordance with one or more embodiments.

FIG. 6 is a flow diagram of a method to implement a physician house call using a physician house call portal in accordance with one or more embodiments.

It will be appreciated that for simplicity and/or clarity of illustration, elements illustrated in the figures have not necessarily been drawn to scale. For example, the dimensions of some of the elements may be exaggerated relative to other elements for clarity. Further, if considered appropriate, reference numerals have been repeated among the figures to indicate corresponding and/or analogous elements.

### DETAILED DESCRIPTION

In the following detailed description, numerous specific details are set forth to provide a thorough understanding of claimed subject matter. It will, however, be understood by those skilled in the art that claimed subject matter may be practiced without these specific details. In other instances, well-known methods, procedures, components and/or circuits have not been described in detail.

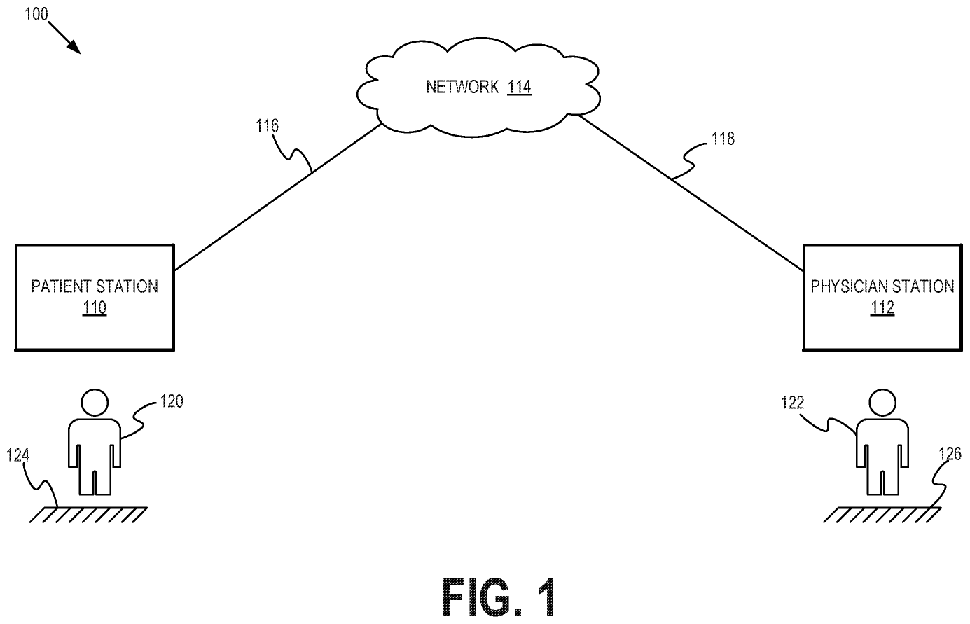

Referring now to FIG. 1, a diagram of a physician house call system including a patient station and a physician station in accordance with one or more embodiments. As shown in FIG. 1, a physician house call system 100 can include a patient station 110 coupled to a physician station 112 via a network 114. The patient station 110 can have a communication link 116 to network 114 wherein communication link 116 can comprise a wired link such as an ETHERNET connection or a wireless link such as a WI-FI link, a cellular network link, a BLUETOOTH or ZIGBEE link, and so on. In some embodiments, communication link 116 can include one or more wired links and one or more wireless links. Physician station 112 can connect to network 114 via communication link 118 which can comprise a wired link, a wireless link, or a combination thereof. In some embodiments, network 114 can include the Internet.

Patient station 110 can be used by a patient 120 located at a first geographic location 124 where the patient 120 is located, and physician station 112 can be used by a medical practitioner 122 such as a doctor, nurse, physician assistant, and so on, located at a second geographical location 126. It is noted that the first geographic location 124 and the second geographic location 126 may be separated by a distance that can be a few meters or can be several kilometers or greater. The physician house call system 100 can allow the medical practitioner 122 to see and evaluate a patient 120 that is not at the same location as the medical practitioner 122. For example, the patient 120 can be at his or her home and the medical practitioner 122 can be at his or her medical office located some distance away from the patient's home. In such an arrangement, the medical practitioner 122 is able to make a house call on the patient 120 without requiring the medical practitioner 112 to be physically at the same location as the patient 120. As discussed herein, a house call can refer to a meeting between a patient 120 and a medical practitioner 122 in which the medical practitioner 122 evaluates the patient 120, although the scope of the disclosed subject matter is not limited in this respect.

In some examples as discussed herein, the physician house call system 100 enables a medical practitioner 122 such as a physician to perform an examination of a remotely located patient 122. Increased efficiency is achieved in some examples through a computer workstation or similar device providing data, voice, and/or video communications with the patient 120 so that the physician or medical practitioner 122 may examine multiple patients per hour without having to travel large distances.

In some examples, patient station 110 can be implemented using a personal computer, mobile device, or a standalone device, which generally can be referred to as a patient computer, and can be configured to be used in the patient's home or another convenient location such as a neighborhood drug store or a local medical clinic. Some examples of such a patient computer can include one or more applications, referred to as one or more patient station applications, that run on the patient computer to enable the patient station 110 to sense and transmit patient data or patient parameters, also referred to as patient medical data, and to communicate with the physician or medical practitioner 122 who is remote from the patient 120 via a communication link such as communication link 116 between patient station 110 and network 114 and/or communication link 118 between physician station 112 and network 114. In some examples the communication link can be configured to support real-time, or near real-time, video and audio communication between the patient 120 and physician or medical practitioner 122 as well as transfer of the patient medical data from the patient station 110 and the physician station 112. In some examples, the communication link can include a cloud-based link in which the communication between the patient station 110 and the physician station 12 is coordinated via a medical server (not shown) as part of network 114. The operation of such a physician house call system 100 is shown in further detail below.

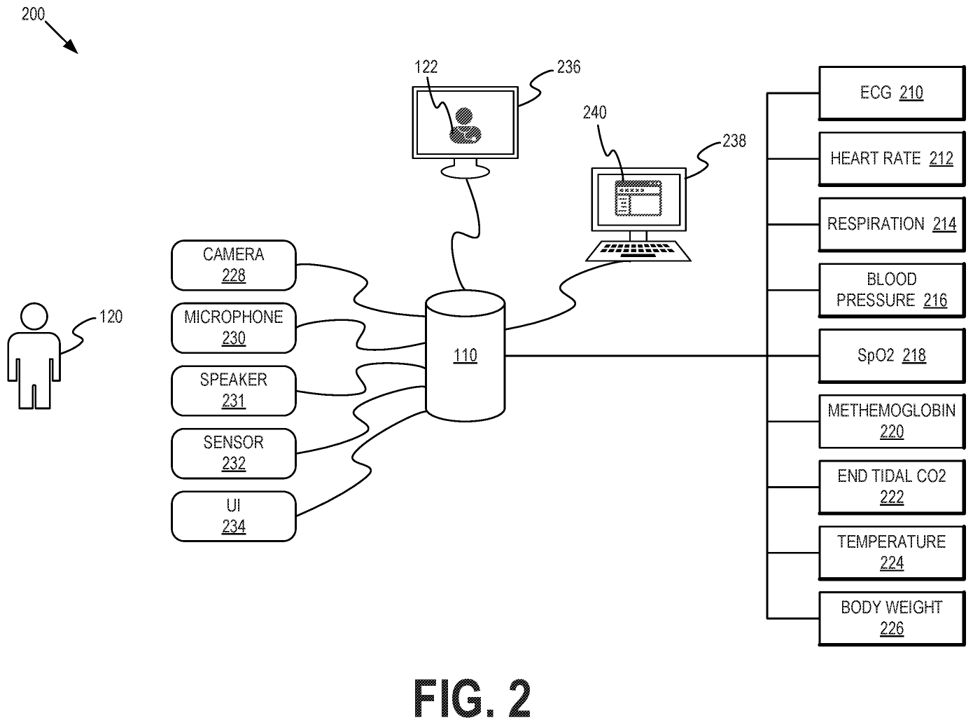

FIG. 2 is a diagram of a physician house call portal including a patient station to obtain one or more patient parameters in accordance with one or more embodiments. As shown in FIG. 2, a physician house call portal 200 can include a patient station 110 as shown in FIG. 1 coupled to a geographically separated physician station 112 via network 114. In some examples, patient station 110 can comprise a small form factor device as shown in FIG. 2 which can be referred to as a peripheral unit or an accessory unit. In such examples, the patient station 110 as a peripheral unit can be preconfigured with one or more patient station applications. In some examples, the peripheral unit has one or more sensors and one or more interfaces to collect one or more patient parameters or patient medical data as described herein below.

In other examples, a patient computer 238 can be provided by the patient 120 and the peripheral unit is coupled to the patient computer 238. In some embodiments the peripheral unit is relatively small and lightweight so that it can be easily shipped or mailed to the patient's home. One or more patient station applications 240 can be stored on a computer readable medium such as a Universal Serial Bus (USB) drive or disk, a Secure Digital (SD) card, a MultiMediaCard (MMC) card, and so on, shipped with the peripheral unit or otherwise obtained separately from the peripheral unit. In some examples, one or more patient applications can come preinstalled on the peripheral unit of the patient station 110. In other examples the patient station applications 240 can be downloaded from the patient station 112 or from a medical server via the communication link 116. The peripheral unit of the patient station 110 and/or the patient computer 238 and one or more patient applications 240 can tangibly embody a house call in a box for patients and doctors or other medical practitioners, family members, or friends of the patient.

In some examples of the patient station 110, the peripheral unit can have sensors for one or more of the following patient physiological parameters: electrocardiogram (ECG) wave forms 210, heart rate 212, respiration rate 214, blood pressure 216, peripheral capillary oxygen saturation (SpO2) 218, methemoglobin 220, end tidal carbon dioxide (CO2), temperature 224, body weight 226, and so on. In some embodiments, these patient parameter sensors can be attached to or integrated into a support structure that the patient 120 wears so that the sensors are disposed appropriately to accurately sense the patient physiological parameters.

In further examples, a camera 228 can be included for physician guided visual examinations of ears, nose, throat, and so on. The camera 228 can be incorporated with an appropriate medical device or instrument such as an otoscope or stethoscope that the patient 120 can manipulate on himself, optionally with guidance from the physician or medical practitioner 122. For example, the physician can guide the patient 120 over communication links 116 and 118 via network 114 in disposing the camera 228 for these examinations and to receive the video data sent back to physician station 112 via network 114. Optionally, a microphone 230 and/or speaker 231 can be provided to allow audio and/or verbal communication between the patient 120 and the medical practitioner 122. In some examples, a display 236 can be included with or otherwise coupled with patient station 110 that can allow the patient 120 to view the doctor or medical practitioner 122, for example to guide the patient through demonstration and/or to otherwise facilitate communication between the physician and patient. In other embodiments, the camera 228 and a light source can disposed in a set of eyeglasses, a headset, or a helmet that the patient 120 wears, and can be configured to be remotely controlled by the physician or medical practitioner 122 to view, for example, one or more of the patient's eyes, mouth, and ears and so on. In some examples, a medical device or instrument can include one or more electronic sensors 232 such as digital stethoscope for auscultation to listen to heart or lung sounds, or a digital otoscope to examine the patient's ears. The peripheral device implementing the patient station 110 can include a user interface (UI) 234 such as a display, touch screen, buttons, keyboard, lights, and/or speakers to allow the patient 120 or another person collocated with the patient 120 to control the patient station 110.

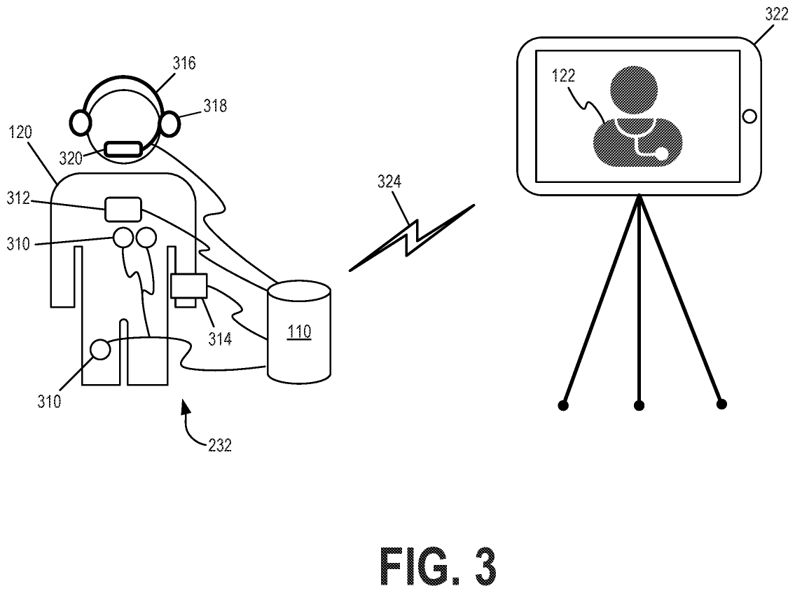

FIG. 3 is a diagram of a physician house call portal with which a patient can view a remotely located medical practitioner in accordance with one or more embodiments. In some examples, the physician station 112 can be implemented in a personal computer, workstation, or mobile device with a web camera and audio head set with an interface to the communication link 118 shown in FIG. 1 above in conjunction with embodiments of a patient station 110. In such examples, the physician station 112 can enable the physician or medical practitioner 122 to remotely perform patient examinations, including receiving and reviewing vital sign measurement taken via the patient station 110, guiding the patient 120 to taken action including positioning sensors 232 such as a camera 228, a light source, a thermometer, a non-invasive blood pressure (NIBP) cuff, stepping on a wired or wirelessly connected scale to measure and forward the patient's weight, to ask the patient questions, and so on. In such examples, the patient 120 can see video of the medical practitioner 122, can hear the medical practitioner, and can speak with the medical practitioner 122. In FIG. 2, the patient station 110 can connect with a personal computer 238. In the example of FIG. 3, the patient station 110 can connect with a mobile device 322 such as a tablet or mobile phone, or any other type of small form factor device or small form factor computer. In some examples, mobile device 322 can be a body worn device such as a watch or other form factor. In some examples, the patient station 110 can be implemented by the personal computer 238 or the mobile device 322 itself without requiring a separate peripheral device as discussed herein.

In some examples, the medical practitioner 122 can communicate with the patient 122 using the mobile device 322. Since the patient can see, hear, and speak with the medical practitioner 122, the medical practitioner 122 can assist or otherwise guide the patient to appropriately place one or more sensors 232 on the patient's body. For example, the patient 120 can be guided where to place one or more ECG sensors 310 on the patient's body, where to place an electronic stethoscope sensor 312 on the patient's body, where to place an NIBP cuff, heart rate, and/or SpO2 sensor 314 on the patients arm or wrist, and so on. In some examples, a sensor 232 can include an ultrasound device. The various sensors 232 can couple with patient station 110 via one or more wired leads or connectors, or can couple with patient station 110 via one or more wireless links such as WI-FI, BLUETOOTH or ZIGBEE or the like. Similarly, patient station 110 can connect with mobile device 322 via one or more wired leads or connectors or one or more wireless links 324. In some examples, the patient 120 can wear headphones or a headset 316 that can include one or more speakers or earmuffs 318 and/or one or more microphones 320 to facilitate communication with the medical practitioner 122 who likewise can optionally wear a headset with a microphone. The patient station 110 can collect the patient parameters from the various sensors 232 and transfer the patient parameter as data sent to the physician station. The patient parameters can be obtained in real-time or near real-time while communicating with the medical practitioner or can be obtained prior to the examination appointment, either independently by the patient 120 or with the support or a nurse or other medical practitioner, a worker, a patient family member, or a patient friend at the patient's location.

In some embodiments, the patient station 110 can be sold or shipped to patients, for example on a permanent ownership basis, or can be provided to patients on a temporary basis as needed then shipped back when no longer needed by the patient 120. In some embodiments, the patient station 110 can be placed at a pharmacy or other retail location that may be more convenient for the patient than traveling to the doctor's office. In such retail deployments, the patient station 110 can be disposed in a booth or other structure to provide privacy for the patient 120 during the remote house call at the retail location.

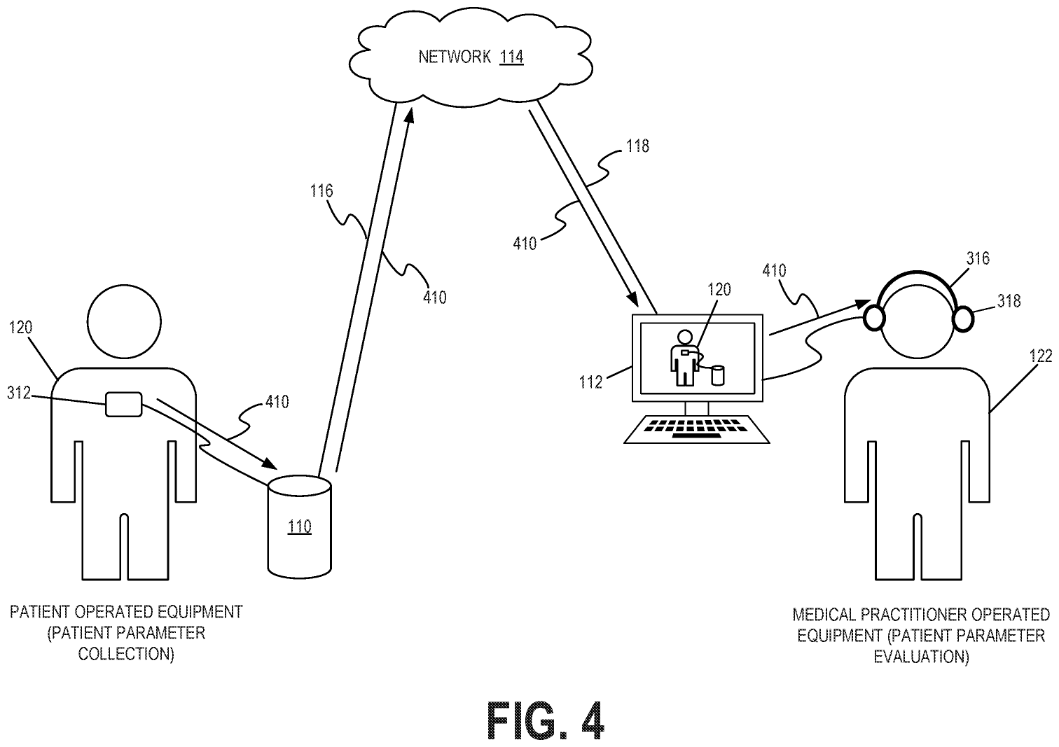

FIG. 4 is a diagram of a physician house call portal with which a medical practitioner can view a remotely located patient in accordance with one or more embodiments. As shown in FIG. 4, a medical practitioner 122 can utilize medical practitioner operated equipment to perform patient parameter evaluation of one or more patient parameters of a remotely located patient 120. Such medical practitioner operated equipment can include a physician station 112. The medical practitioner 122 is able to communicate with the patient 120 via network 114 and can view the patient 120 to determine whether the patient 120 is properly operating the patient operated equipment including the patient station 110 and to guide the patient 120 for proper patient parameter collection.

For example, the patient 120 may be using a patient operated electronic stethoscope 312 so that the medical practitioner 122 can hear and monitor the auscultation of the patient's heart or the patient's lungs. The auscultations can be converted to patient parameter data 410, for example as a digital audio signal, that is transmitted from the stethoscope 312 to the patient station 110, from the patient station 110 to the network 114 via communication link 116, from network 114 to physician station 112 via communication link 118, and then to a speaker 318 of a headset 316 worn by the medical practitioner 318. The medical practitioner 122 can thus hear the auscultations of the heart of a remotely located patient 120 in real-time or near real-time.

In addition, using the physician house call system, the medical practitioner is capable of guiding and assisting the patient 312 to perform the patient parameter collection on himself or herself as if the medical practitioner 122 were in the same room as the patient 120 without requiring the assistance of a trained medical person at the patient's location, while also ensuring that the patient parameter collection is properly performed. For example, if the patient 120 was holding the stethoscope 312 in the wrong location or at a non-ideal location, the medical practitioner could view this situation via the display of the physician station 112 and could guide the patient 120 to move the stethoscope 312 to the correct location by providing verbal commands to the patient 120 until the medical practitioner 122 can see that the stethoscope 312 has been placed in the right location on the patient's body. Thus, the physician house call system 100 as shown in and described with respect to FIG. 1 and the various other figures can enable a physician or other medical practitioner 122 to perform a virtual house call on the patient 120 without compromising the quality of the medical evaluation of the patient 120 and also while facilitating with the patient in self-directed patient parameter collection with the assistance of a trained physician or medical practitioner 122. Thus, the physician house call system 100 as described herein is capable of transmitting one-way or two-way communications between the medical practitioner 122 and the patient 120 including audio and/or video along with the patient parameter data in real-time or near real-time. Near real-time can refer to communications in which there is a slight lag or delay in the communications due to packet streaming or other slight transmission delay but is otherwise considered as “live” communications, although the scope of the disclosed subject matter is not limited in this respect.

In some embodiments the physician house call system 100 and/or one or more components thereof can be configured to support multiple way video and audio communications so the patient's advocate such as a caretaker, family member, friend, nurse, and so on can also participate in the house call, for example using a personal computer or mobile device of the advocate who can also be located in a third location at some distance away from the locations of either the patient 120 or the medical practitioner 122. In some embodiments, another party or entity such as the manufacturer or distributor of the patient station 110 or another service provider can provide patient support to the patient 120 prior to the house call from the physician or medical practitioner 122. For example, such another party can guide the patient 120 in a step by step way to measure and capture certain patient physiological data or patient parameters. Some embodiments can involve collecting the patient physiological data and other patient data, performing an initial screening or analysis of the patient data, and notifying the doctor or medical practitioner 122 when the patient data is ready for review by the doctor or medical practitioner 122. In some embodiments, this initial processing can be done via artificial intelligence (AI) based diagnostics. In some embodiments, such another party or entity can perform these processes via a medical server via network 114 and/or using physician station 112 as described herein above. In other embodiments, the patient support functionality can be implemented in an application 240 running on the patient computer 238, on the mobile device 322, or on the patient station 110 as described herein above

In one or more embodiments, patient station 110 can be mailed or shipped to the patient 120 as a kit including one or more devices or medical sensors that the patient 120 can set up and configure at the patient's home. The patient 120 can be walked the through the usage and placement of the devices and sensors on the patient's body, and once the patient 120 has successfully set up the patient station 110, obtaining one or more patient physiological parameters can occur and a connection can be made with the physician station 112 to begin the physician house call or telemedicine. In some examples, the patient station 110 can be used to assist the patient 120 in configuring the patient station 110 and the one or more sensors or medical devices. For example, a video can be played on a display such as display 236 that provides guidance and illustrations for the patient 120 on how to properly place and use the one or more sensors or medical devices. In some examples, an augmented reality (AR) or mixed reality (MR) headset can provided that the patient 120 can wear to help guide the patient 120 through the process of setting up the patient station 110 and verifying that everything is set it up correctly. In some examples, the camera 228 and/or display 236 of the patient station can functions as an AR or MR headset, for example via an attachment to a headband, headset, headgear, or helmet to assist the patient 120 in wearing and using the AR or MR features. In some examples, the mobile device 322 can be used as the AR or MR headset by attaching the mobile device 322 to a suitable head worn device. In some examples, the AR or MR features can be provided by a commercially available AR or MR type device such as a HoloLens device available from Microsoft Corporation or an Oculus type device available from Facebook Technologies, LLC. It should be noted that these are merely some examples of how a patient can use AR or MR features to assist with setting up a patient station 110, and the scope of the disclosed subject matter is not limited in these respects.

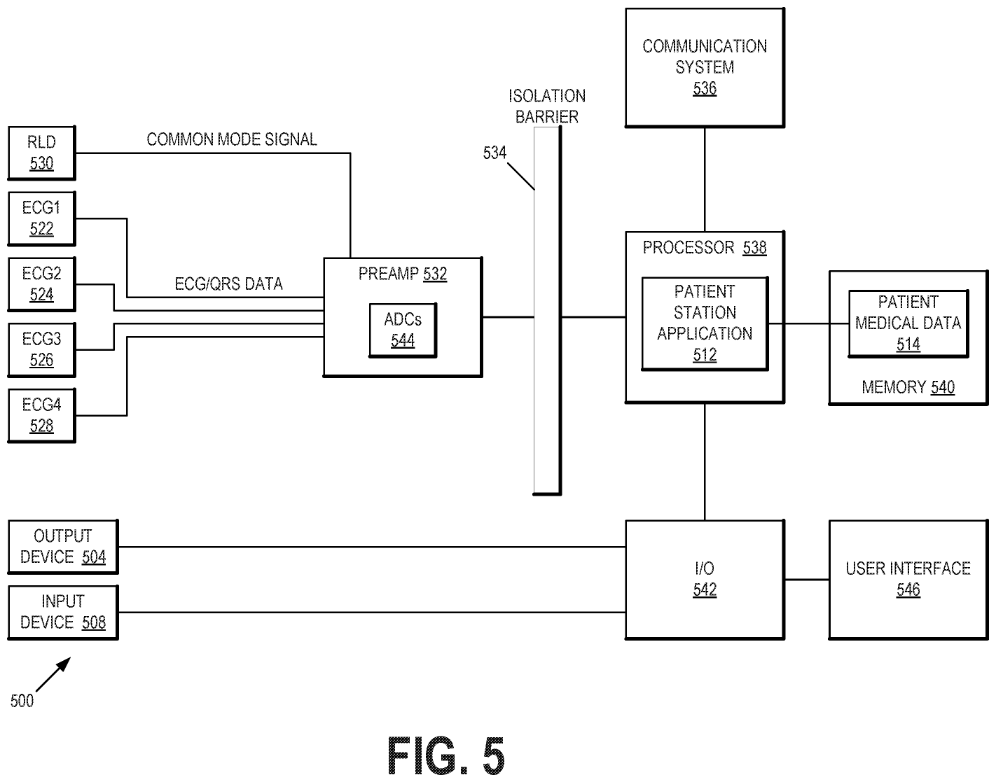

FIG. 5 is a diagram of example components of a physician house call portal including electrocardiogram (ECG) sensors in accordance with one or more embodiments. The physician house call portal 500 of FIG. 5 can include components to obtain ECG and QRS complex signal data detection along with heart rate data detection. In one or more examples, the physician house call portal 500 of FIG. 5 can comprise a single device, one or more components, a system comprising one or more subsystems, a system comprising one or more devices, or a system that comprises one or more devices and/or one or more subsystems and/or one or more components, and the scope of the disclosed subject matter is not limited in these respects. In some examples, some or all of the components of a physician house call portal 500 can be incorporated into a patient station 110 as discussed herein, for example where the patient station 110 comprises a peripheral device or other form factor including patient computer 238 or mobile device 322. Furthermore, the physician house call portal 500 can comprise a single device or physician house call system 100 as shown in FIG. 1, where a system can comprise one or more components, a system can comprise one or more subsystems, a system can comprise one or more devices, or a system can comprise one or more devices and/or one or more subsystems and/or one or more components, and the scope of the disclosed subject matter is not limited in these respects. In the example shown in FIG. 5, physician house call portal 500 can include one or more components and/or one or more subsystems of physician house call system 100 as discussed herein and as shown in FIG. 1 and so on. Furthermore, the elements of the physician house call portal 500 of FIG. 5 can comprise or include one or more of the components of any of the figures herein. In addition, the physician house call portal 500 optionally can include or otherwise can operate cooperatively with one or more small form factor wearable medical devices or sensors 232 such as ECG sensors 310, stethoscope 312, NIBP cuff, heart rate, and/or SpO2 sensor 314 as shown in FIG. 3, and the scope of the disclosed subject matter is not limited in this respect.

The physician house call portal 500 can include ECG electrodes, ECG1 522, ECG2 524, ECG3 526, and ECG4 528, that can be implemented in a number of ways. One such way is by using silver or silver plated copper electrodes that dry attach to the skin of the patient 120. The ECG electrodes provide ECG/QRS data to preamplifier 532. The preamplifier 532 may have a wide dynamic range at its input, for example +/−1.1 V which is much larger than the amplitude of the ECG signals which are about 1 mV. The preamplifier 532 includes analog-to-digital converters (ADCs) 544 to convert the ECG signals into a digital format. A right-leg drive (RLD) electrode 530 can be used to provide a common mode signal so that the ECG signal from the ECG electrodes may be provided to preamplifier 532 as differential signals. The digital ECG signals are provided from the preamplifier 532 eventually to the main processor 538 via an optional isolation barrier 534. When provided, isolation barrier 534 operates to electrically isolate the preamplifier 532 and the ECG signals from the rest of the circuitry of the physician house call portal 500. In some examples, isolation barrier 534 can include one or more optocouplers and/or transformers to provide the electrical isolation.

The processor 538 can be used to run one or more patient station applications 512 such as the patient station applications 240 shown in FIG. 2, and can store one or more collected patient parameters as patient medical data 514 in memory 540. The patient 120 can control the operation of the physician house call portal 500 with a user interface (U/I) 546 coupled to processor 538 via an input/output (I/O) system 542 wherein the UI 546 can comprise UI 234 of FIG. 2. The I/O system 542 can couple with one or more output devices 504 and one or more input devices 508. The output devices 504 can include a display 236, a speaker 231, earmuff 318, a light, and so on as shown in and described with respect to FIG. 2 and FIG. 3. Similarly, the input devices 508 can include camera 228, microphone 230, sensor 232, ECG sensors 310, stethoscope 312, NIBP cuff, heart rate, and/or SpO2 sensor 314, microphone 320, and so on as shown in and described with respect to FIG. 2 and FIG. 3 including one or more of patient parameter sensors for obtaining any one or more of the patient physiological parameters 210 through 226 shown in FIG. 2. In addition, the physician house call portal 500 can include a communication system 536 that may include one or more wired communication circuits such as ETHERNET or Universal Serial Bus (USB), and/or one or more wireless communication circuits such as BLUETOOTH, ZIGBEE, WI-FI, or cellular radio-frequency (RF) circuits including supporting circuitry such as one or more baseband processors and one or more transceivers and antennas.

In accordance with one or more embodiments, the physician house call portal 500 can include an SpO2 sensor 218 and/or temperature sensor 224 that can be coupled to the preamp 532. In some embodiments, patient impedance measurements may be obtained between any two or more of the ECG electrodes, for example to determine a patient's respiration. In other embodiments, the physician house call portal 500 can comprise a wearable patient monitoring system that is capable of collecting one or more patient parameters that can be stored in a memory 540 for future review and analysis, and/or to provide one or more warnings to a patient that one or more patient parameters are outside a normal or predetermined range when the patient is wearing the patient monitoring system, for example to allow the patient to cease a present activity that may be causing an atypical patient parameter or to otherwise seek assistance or medical help from the medical practitioner 122 or other persons. In one or more embodiments, any one or more of the components or functions of the physician house call portal 500 can be embodied as hardware, software, and/or firmware, for example as instructions stored on a machine or computer readable medium that when executed by a processor implement one of more functions of the physician house call portal 500. Although FIG. 5 shows the patient station application 512 operating on processor 538, it should be noted that one or more other electronic and/or software elements of the physician house call portal 500 can include their own processor, memory, and one or more applications that can work independently of or in cooperation with the physician house call portal 500. In some embodiments, memory 540 and patient medical data 514 can be located external to processor 538 and/or external to the physician house call portal 500, and in other embodiments, memory 540 and patient medical data 514 can be located internal to processor 538.

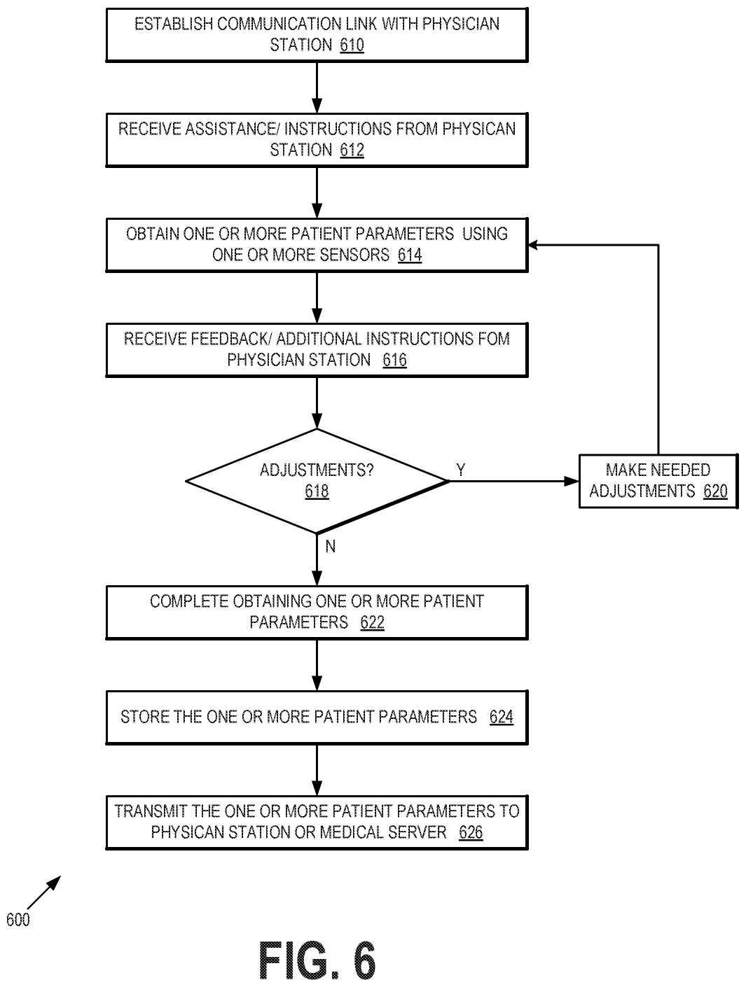

Referring now to FIG. 6, a flow diagram of a method to implement a physician house call using a physician house call portal in accordance with one or more embodiments will be discussed. Method 600 of FIG. 6 can be performed by physician house call portal 500 of FIG. 5, for example using a physician station 110, one or more patient station applications 240, patient computer 238, mobile device 322, processor 538, and/or patient station application 512. It should be noted that FIG. 6 illustrates merely one example of method 600, and method 600 may include more or fewer blocks in various other orders, and the scope of the disclosed subject matter is not limited in these respects.

At block 610, a communication link can be established with physician station 112, for example by having patient station 110 initiate communicate with physician station 112, or by having physician station 112 communicate with patient station 110. After establishing a communication link, assistance and/or instructions can be received from physician station 112 at block 612. For example, the medical practitioner 122 can give audio and/or visual instructions to the patient 120 using the physician house call portal 500 instructions regarding which sensors 232 or other medical devices to use, how to use the devices or sensors, and/or where on the patient's body to place the sensors 232. The medical practitioner 122 can also send electronic instructions and/or commands to the patient station 110 via the physician station 112 such that the medical practitioner 122 can at least partially control the one or more sensors 232 of the physician house call portal 500. At block 614, one or more patient parameters can be obtained using one or more of the sensors 232 at block 614. The one or more patient parameters optionally can be transmitted to the physician station 112 at this time for monitoring and review by the medical practitioner 112, for example where the one or more patient parameters are transmitted to physician station 112 in real-time or near real-time in a “live” parameter monitoring situation. In some examples, the medical practitioner 122 can monitor and review the patient's actions using camera 228 and/or microphone 230 for example as shown in FIG. 4. Such an arrangement can allow the medical practitioner 122 and/or the physician station 112 to provide feedback and/or additional instructions to the physician house call portal 500 at block 616. For example, the feedback or additional instructions can instruct the patient 120 to move the sensor 232 to a different location of the patient's body, for example to obtain a better reading or sensing of the patient parameter or to obtain a new patient parameter reading.

At decision block 618, a determination can be made whether one or more adjustments need to be made, for example to adjust the sensor 232 or to change one or more settings of the sensor 232 or the physician house call portal 500. If one or more adjustments are needed, the adjustments can be made at block 620, and method 600 can continue at block 614. If no adjustments or needed, or no further adjustments are needed, obtaining one or more patient parameters can be completed at block 622, and one or more of the patient parameters can be stored at block 624. The one or more patient parameters can be transmitted to the physician station or a medical server at block 626, for example for later review by the medical practitioner 122 and/or for record keeping or archiving purposes.

In one or more alternative embodiments, instead of first establishing a link with the physician station 112 as shown at block 610, the patient 120 may first connect one or more sensors 232 of the patient station 110 to the patient's body in order to acquire a baseline measurement of one or more patient physiological parameters prior to establishment of the communication link. Some patient physiological parameters may be easier for the patient 120 to obtain himself or herself in a self-directed manner without requiring assistance or instructions to obtain the measurements, or once the patient 120 has been instructed how to perform the measurements that may be more involved. For example, the patient 120 can step on a scale connected to the patient station 110 to get a weight reading, can use a thermometer connected to the patient station 110 to get a temperature reading, and so on. In some examples, the patient 120 can also have the ability to obtain a heart rate measurement, a blood pressure measurement, an SpO2 measurement, and so on, in a self-directed manner without assistance or instructions from another person. In some examples, an aid, friend, family member, or other assistant or medical practitioner at the patient's location could be available to assist with obtaining one or more pre-house call patient physiological parameter measurements. In such an arrangement, the baseline measurement for one or more of the patient physiological parameters can be ready and already available at the time of the communication link is made, thereby saving time and providing efficiency to the physician house call. Other various embodiments and orders of method 600 may likewise be implemented, for example depending on the level of skill or training of the patient 110 to perform self-directed measurements, and the scope of the disclosed subject matter is not limited in this respect.

In one or more embodiments, the physician house call portal as discussed herein can be adapted to be deployed in a healthcare environment such as a clinic, emergency room (ER), and so on. In such embodiments, each patient 120 can be provided with a patient station 110, the patient station 110 could already be installed in each patient room or area or could be located in a waiting room or in a pre-examination area. In some examples, the patient 110 could bring his or her own personal patient station 110 if the patient already has a suitable one. A collection of multiple patient stations 110 could communicate with a medical server of network 114 for coordination of the multiple stations, and/or to upload any collected patient physiological parameter measurements to a storage location or to the physician station 112. While one or more of the patients 120 are waiting to be seen by a medical practitioner 122, either directly or remotely via a physician station 112, one or more patient physiological parameters may be monitored by the respective patient stations 110 so that a baseline set of measurements can be obtained for each patient prior to the physician meeting, for example as discussed herein above. Furthermore, in some examples the patient physiological parameter measurements can be used as part of a set of triage criteria to determine the order in which multiple patients at the clinic or ER should be examined and/or treated.

In some examples, multiple patient stations 110 located at each of the patient's homes or other remote locations can similarly communicate simultaneously, or nearly simultaneously or within a relatively short period of time, with a physician station 112. Triage criteria can use the collected patient physiological parameter measurements obtained from each of the multiple patient stations 110 so that a determination can be made of which order a physician house call should be made for each of the patients. In some examples, the medical practitioner 122 can review the measurements to determine the order in which the patients should be called on, or alternatively a program or application running on physician station 112 can analyze the measurements to make this decision or suggest an order to the medical practitioner 112. In some examples, one or more of the patients can be located at a home location, one or more of the patients can be located at a clinic or a retail location, and one or more of the patients can be located in a doctor's office or at a hospital, and so on, and the scope of the disclosed subject matter is not limited in this respect.

The following are example implementations of the subject matter described herein. It should be noted that any of the examples and the variations thereof described herein may be used in any permutation or combination of any other one or more examples or variations, although the scope of the claimed subject matter is not limited in these respects. In example one, a physician house call portal comprises a processor and a memory coupled to the processor to store a patient station application, a communication system to communicate with a physician station via a network, an input/output (I/O) system coupled with an input device and an output device to allow a patient to communicate with a medical practitioner using the physician station, and a medical sensor to obtain a patient physiological parameter using the patient station application. Instructions in the memory configure the processor to allow the medical practitioner to perform an examination of the patient using the medical sensor. In example two, the processor is configured to provide control of the medical sensor to the medical practitioner via the physician station. In example three, the patient is capable of receiving instructions from the medical practitioner via the physician station on operating the medical sensor. In example four, the instructions in the memory are to cause the processor to transmit the patient physiological parameter to the physician station in real-time or near real-time. In example five, the input device includes a camera and a microphone. In example six, the output device includes a display and a speaker. In example seven, the patient is to receive guidance from the medical practitioner regarding obtaining the patient physiological parameter using the medical sensor. In example eight 8, the medical sensor comprises a plurality of electrocardiogram (ECG) electrodes coupled with a preamplifier to obtain ECG signals from the patient as the patient physiological parameter. In example nine, the medical sensor comprises a blood pressure sensor. In example ten, the medical sensor comprises a stethoscope or an otoscope.

In example eleven, a patient station comprises one or more medical device sensors to obtain one or more patient parameters from a patient, a camera to allow a medical practitioner to observe the patient regarding the one or more medical device sensors obtaining the one or more patient parameters, and a user interface to allow instructions to be received regarding the use of the one or more medical device sensors, wherein the instructions indicate one or more adjustments to be made regarding the one or more medical device sensors obtaining the one or more patient parameters. In example twelve, the user interface comprises a speaker to provide audio instructions regarding the one or more medical device sensors obtaining the one or more patient parameters. In example thirteen, the adjustments are to be implemented by the patient. In example fourteen, the adjustments are to be implemented without requiring action by the patient. In example fifteen, the patient station further comprises an input/output system to couple the patient station to a patient computer.

In example sixteen, a non-transitory computer readable medium has instructions stored thereon that when executed result in establishing a communication link with a remotely located physician station, receiving instructions from the physician station regarding obtaining one or more patient parameters from a patient using one or more sensors, obtaining the one or more patient parameters from the patient using the one or more sensors, and transmitting the one or more patient parameters to the physician station. In example seventeen, the communication link is made via a network. In example eighteen, the instructions, when executed, further result in transmitting a video stream of the patient to the physician station showing the patient during said obtaining. In example nineteen, the instructions, when executed, further result in receiving feedback from the physician station whether any adjustments should be made regarding said obtaining, and if so making one or more adjustments. In example twenty, the instructions, when executed, further result in storing the one or more patient parameters in a memory.

In example twenty-one, a method, for use by a medical practitioner in providing instructions to a remotely-located patient and receiving medical information from the remotely-located patient, comprises providing a patient station to be delivered to a remotely-located patient, the patient station including one or more medical device sensors to obtain one or more patient parameters from a patient, a camera to enable a medical practitioner to observe the patient regarding the one or more medical device sensors obtaining the one or more patient parameters, and a user interface to enable instructions to be received regarding the use of the one or more medical device sensors, wherein the instructions indicate one or more adjustments to be made regarding the one or more medical device sensors obtaining the one or more patient parameters, establishing a communication link with the remotely located patient station, providing instructions to the remotely-located patient, via the communication link and the user interface, regarding a set-up of the patient station and/or operation of the patient station, causing the remotely-located patient station to provide instructions regarding obtaining one or more patient parameters from a the remotely-located patient using the one or more medical device sensors, and receiving, via the communication link, from the remotely-located patient station the one or more patient parameters obtained from the remotely-located patient using the one or more medical device sensors. In example twenty-two, the communication link is made via a network. In example twenty-three, receiving the one or more patient parameters comprises receiving a video stream of the patient. In example twenty-four, said providing instructions to the remotely-located patient further comprises providing augmented reality (AR) and/or mixed reality (MR) assisted instructions by the medical practitioner to the remotely-located patient via the user interface, the user interface comprising a MR or VR headset. In example twenty-five, the camera of the patient station includes a display and is configured to be removably attached to the MR or VR headset to enable the remotely-located patient to view the display and orient the camera in the direction in which the remotely-located patient is looking.

In the description herein and/or claims, the terms coupled and/or connected, along with their derivatives, may be used. In particular embodiments, connected may be used to indicate that two or more elements are in direct physical and/or electrical contact with each other. Coupled may mean that two or more elements are in direct physical and/or electrical contact. Coupled, however, may also mean that two or more elements may not be in direct contact with each other, but yet may still cooperate and/or interact with each other. For example, “coupled” may mean that two or more elements do not contact each other but are indirectly joined together via another element or intermediate elements. Finally, the terms “on,” “overlying,” and “over” may be used in the following description and claims. “On,” “overlying,” and “over” may be used to indicate that two or more elements are in direct physical contact with each other. It should be noted, however, that “over” may also mean that two or more elements are not in direct contact with each other. For example, “over” may mean that one element is above another element but not contact each other and may have another element or elements in between the two elements. Furthermore, the term “and/or” may mean “and”, it may mean “or”, it may mean “exclusive-or”, it may mean “one”, it may mean “some, but not all”, it may mean “neither”, and/or it may mean “both”, although the scope of claimed subject matter is not limited in this respect. In the description herein and/or claims, the terms “comprise” and “include,” along with their derivatives, may be used and are intended as synonyms for each other.

Although the claimed subject matter has been described with a certain degree of particularity, it should be recognized that elements thereof may be altered by persons skilled in the art without departing from the spirit and/or scope of claimed subject matter. It is believed that the subject matter pertaining to physician house call portal and many of its attendant utilities will be understood by the forgoing description, and it will be apparent that various changes may be made in the form, construction and/or arrangement of the components thereof without departing from the scope and/or spirit of the claimed subject matter or without sacrificing all of its material advantages, the form herein before described being merely an explanatory embodiment thereof, and/or further without providing substantial change thereto. It is the intention of the claims to encompass and/or include such changes.

What is claimed is:

1. A physician house call portal, comprising:
a processor and a memory coupled to the processor to store a patient station application;
a communication system to communicate with a physician station via a network;
an input/output (I/O) system coupled with an input device and an output device to allow a patient to communicate with a medical practitioner using the physician station; and
a medical sensor to obtain a patient physiological parameter using the patient station application;
wherein instructions in the memory configure the processor to allow the medical practitioner to perform an examination of the patient using the medical sensor.

2. The physician house call portal of claim 1, wherein the processor is configured to provide control of the medical sensor to the medical practitioner via the physician station.

3. The physician house call portal of claim 1, wherein the patient is capable of receiving instructions from the medical practitioner via the physician station on operating the medical sensor.

4. The physician house call portal of claim 1, wherein the instructions in the memory are to cause the processor to transmit the patient physiological parameter to the physician station in real-time or near real-time.

5. The physician house call portal of claim 1, wherein the input device includes a camera and a microphone.

6. The physician house call portal of claim 1, wherein the output device includes a display and a speaker.

7. The physician house call portal of claim 1, wherein the patient is to receive guidance from the medical practitioner regarding obtaining the patient physiological parameter using the medical sensor.

8. The physician house call portal of claim 1, wherein the medical sensor comprises a plurality of electrocardiogram (ECG) electrodes coupled with a preamplifier to obtain ECG signals from the patient as the patient physiological parameter.

9. The physician house call portal of claim 1, wherein the medical sensor comprises a blood pressure sensor.

10. The physician house call portal of claim 1, wherein the medical sensor comprises a stethoscope or an otoscope.

11. A patient station, comprising:
one or more medical device sensors to obtain one or more patient parameters from a patient;
a camera to allow a medical practitioner to observe the patient regarding the one or more medical device sensors obtaining the one or more patient parameters; and
a user interface to allow instructions to be received regarding the use of the one or more medical device sensors, wherein the instructions indicate one or more adjustments to be made regarding the one or more medical device sensors obtaining the one or more patient parameters.

12. The patient station of claim 11, wherein the user interface comprises a speaker to provide audio instructions regarding the one or more medical device sensors obtaining the one or more patient parameters.

13. The patient station of claim 11, wherein the adjustments are to be implemented by the patient.

14. The patient station of claim 11, wherein the adjustments are to be implemented without requiring action by the patient.

15. The patient station of claim 11, further comprising an input/output system to couple the patient station to a patient computer.

16. A non-transitory computer readable medium having instructions stored thereon that when executed result in:
establishing a communication link with a remotely located physician station;
receiving instructions from the physician station regarding obtaining one or more patient parameters from a patient using one or more sensors;
obtaining the one or more patient parameters from the patient using the one or more sensors; and
transmitting the one or more patient parameters to the physician station.

17. The non-transitory computer readable medium of claim 16, wherein the communication link is made via a network.

18. The non-transitory computer readable medium of claim 16, wherein the instructions, when executed, further result in transmitting a video stream of the patient to the physician station showing the patient during said obtaining.

19. The non-transitory computer readable medium of claim 16, wherein the instructions, when executed, further result in receiving feedback from the physician station whether any adjustments should be made regarding said obtaining, and if so making one or more adjustments.

20. The non-statutory computer readable medium of claim 16, wherein the instructions, when executed, further result in storing the one or more patient parameters in a memory.
